(19) United States
(12) Patent Application Publication
(10) Pub. No.: US 2018/0199531 A1
(43) Pub. Date: Jul. 19, 2018

(54) Specialty Breeding of Cannabis

(71) Applicant: John Michael Dawson, II, Vashon Island, WA (US)

(72) Inventor: John Michael Dawson, II, Vashon Island, WA (US)

(21) Appl. No.: 15/868,053

(22) Filed: Jan. 11, 2018

Related U.S. Application Data
(60) Provisional application No. 62/446,939, filed on Jan. 17, 2017.

(51) Int. Cl. A01H 5/12

(52) U.S. Cl. CPC A01H 5/12

(57) ABSTRACT

The invention provides compositions and methods for a specialty breeding of cannabis.

### FIELD

The instant application relates to a specialty breeding of cannabis plant.

### BACKGROUND

Cannabis, more commonly known as marijuana, is a genus of flowering plants that includes species Cannabis sativa, Cannabis indica, and Cannabis ruderalis as determined by plant phenotypes and secondary metabolite profiles.

The use of cannabis for social and medical purposes is well known. Cannabis is most commonly administered via inhalation or consumption of marijuana-infused food and drink. However, since 1972 marijuana has been classified as a Schedule I drug under the U.S. Controlled Substances Act because the U.S. Federal Government considers it to have “no accepted medical use.” In contrast to this position, 33 of the 50 U.S. states and the District of Columbia have recognized the medical benefits of cannabis and have decriminalized its medical use. The 33 U.S. states were medical marijuana has been decriminalized at the current time are as follows: Alaska, Arizona, Arkansas, California, Colorado, Connecticut, Delaware, Florida, Hawaii, Illinois, Louisiana, Maine, Maryland, Massachusetts, Michigan, Minnesota, Mississippi, Missouri, Montana, Nevada, North Dakota, North Carolina, Nebraska, New York, New Hampshire, New Jersey, New Mexico, Ohio, Oregon, Pennsylvania, Rhode Island, Vermont and Washington. The residency requirements, approved list of conditions/diseases, and the other laws/rules regarding the possession and cultivation of medical marijuana generally differ state by state.

In addition to these recent developments, the U.S. government has already set a precedent for patenting cannabis, and cannabis-related inventions. For example, U.S. Pat. No. 6,630,507 issued on Oct. 7, 2003 and assigned on the patent face to The United States of America, is directed to methods of treating diseases caused by oxidative stress by administering therapeutically effective amounts of a cannabidiol (CBD) cannabinoid from cannabis that has substantially no binding to the N-methyl-D-aspartate (NMDA) receptor, wherein the CBD acts as an antioxidant and neuroprotectant. A search of the U.S.P.T.O. Patent Application Information Retrieval (PAIR) system also reveals the existence of thousands of cannabis related applications and issued patents including U.S. Pat. No. 8,034,843 (use of cannabinoids for treating nausea, vomiting, emesis, motion sickness), U.S. Pat. No. 7,698,594 (cannabinoid compositions for treatment of pain), and U.S. Pat. No. 8,632,825 (anti-tumoural effects of cannabinoid combinations) among many others.

On Aug. 4, 2015, U.S. Pat. No. 9,095,554 issued claiming a breeding, production, processing and use of specialty cannabis. In claim 1, the patent claims a hybrid cannabis plant.

Thus, despite the official position of the U.S. Federal Government, and as recognized by the states that have legalized it, cannabis has been shown to provide substantial benefits for medical and recreational uses. Cannabis is regularly used by a wide cross-section of society to treat a variety of maladies, conditions and symptoms including, but not limited to, the following: insomnia, mood swings, anxiety, lack of appetite, inflammation, (non-addictive) pain management, spastic bladder, COPD (chronic obstructive pulmonary disease), TBI (traumatic brain injury), macular degeneration, Parkinson's, fibromyalgia, tumors, lesions, seizures, post-traumatic stress disorder, and tooth infections.

Cannabis intoxication (i.e., euphoria, relaxation) can occur and other side effects may also accompany its use, particularly with higher doses, specific cannabis varieties and/or over prolonged periods of usage. Undesirable side effects of using cannabis varieties can include, but are not limited to, the following: decreased short-term memory, dry mouth, impaired visual perception and motor skills, lower fertility, red (i.e. blood shot) eyes, increased anxiety, paranoia, acute psychosis, lowered mental aptitude, hallucinations, bizarre behavior, irrational panic attacks, irrational thoughts and various other cognitive and social problems.

Some of the effects from using available cannabis varieties for medical and recreational purposes are related to the plant's content of the chemical Δ9-tetrahydrocannabinol (THC). A major hurdle to the more wide-spread acceptance of cannabis and its legalization is that the land races and commercially available cannabis genotypes (of drug varieties) contain relatively high concentrations of THC. Indeed the average THC content of traditional recreational cannabis has risen over the years from an average of 0.74 in 1975, to 3.35% in the 1990's, and average of 6.4% in 2003 (Annual Reports (Nov. 9, 1999 to Nov. 8, 2003) of Mahmoud A. El Sohly, PhD, Director of the National Institute on Drug Abuse (NIDA) Marijuana Project at the National Center for Natural Products Research, School of Pharmacy, University of Mississippi). There is a real need for cannabis varieties for potential medical use that produce modulated THC concentrations and varying concentrations of other pharmacologically active substances that reduce the negative side effects of THC and increase the medical benefits realized from its use. There is also a need for healthier cannabis for recreational use with reduced negative side effects from THC. The inventions described herein meet that long-felt need.

### SUMMARY OF THE INVENTION

The instant application claims the priority of the provisional application No. 62/4,446,939.

According to the compositions of the instant application, plants, plant parts, plant tissues and plant cells are produced to contain cannabigerolic acid, cannabigerol, tetrahydrocannabinolic acid, tetrahydrocannabinol, delta-9-tetrahydrocannabinolic acid, delta-9-tetrahydrocannabinol, cannabinol, cannabidiolic acid, cannabidiol, cannabichromene; and, cis- and trans-terpenoids, including but not limited to linalool, caryophyllene oxide, myrcene, beta-pinene, limonene, terpinolene, alpha-pinene, humulene, and caryophyllene, ranging from less than 0.1% of dry weight of inflorescences, plant parts, plant tissues and plant cells to 14% of inflorescences and/or 14% of plant parts, plant parts, plant tissues and plant cells.

The instant application provides specialty cannabis plants, plant parts, plant tissues and plant cells which provide a way to deliver a consistent and more tolerable and effective ratio of cannabinoids by providing plants that comprise of a low non-THC cannabinoids (“CBs”) to patients.

The instant application provides specialty cannabis plants, plant parts, plant cells and plant tissues which have an amount, percentage and/or ratio of cannabinoids that is different from currently available THCA/THC varieties.

In some embodiments, the instant application provides specialty cannabis plants, plant parts, tissues and cells having a total THC content that is 2.0% but 13% based on the dry weight of plant inflorescences; and, a non-THC CB total content based on the dry weight of plant inflorescences that is ≤9.5%. Thus, the specialty cannabis plants, plant parts, plant tissues and plant cells of the present invention will have a THC content selected from the group consisting of 2%, 3%, 4%, 5%, 6%, 7%, 8%, 9%, 10%, 11%, 12%, and 13% ; and, a CBs content selected from the group consisting of 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3%, 4%, 5%, 6%, 7%, 8%, 9% and 9.5%.

In some embodiments, the instant application provides specialty cannabis plants, plant parts, tissues and cells having a THC:CBs ratio greater than or equal to of 1.3:1. In other embodiments, the specialty cannabis of the present invention has THC:CBs ratios approaching 1:1, or lower. By comparison, the THC:CBs ratio of the currently available cannabis varieties is 20:1 and approaches 25:1, 30:1, 35:1, 40:1 and higher. Thus, the specialty cannabis plants, plant parts, plant tissues and plant cells of the present invention will have a THC:CBs ratio of less than 2:1, 1:1, 1:2, 1:3, 1:4, 1:5, 1:6, 1:7, 1:9, or below.

The instant application provides classes of cannabis varieties developed by selection from landraces of mixed cannabis genotypes and resulting from further breeding, wherein these classes of cannabis varieties can provide useful patient treatment and also are used as breeding material to develop specialty cannabis plants and varieties according to the instant application.

### DETAILED DESCRIPTION

In some embodiments, the instant application teaches a cannabis plant, plant part, tissue, or cell comprising: a cannabidiol (CBD) content that is greater than 0.1% by weight, and a terpene profile which may comprise linalool, caryophyllene oxide, myrcene, beta-pinene, limonene, terpinolene, alpha-pinene, humulene, and caryolphyllene wherein the cannabinoid and terpene content is measured by high performance liquid chromatograph (“HPLC”) and calculated based on dry weight of the inflorescence.

In some embodiments, the cannabis plant, plant part, tissue or cell of the instant application comprises a tetrahydrocannabinol (THC) content that is at least 1.0% by weight as measured by HPLC and calculated based on dry weight of the inflorescence.

In some embodiments, the cannabis plant, plant part, tissue, or cell of the present invention comprises at least 0.03% cannabichromene (CBC) content by weight.

In some embodiments, the cannabis plant, plant part, tissue or cell of the instant application comprises a total CBD content that is at least 5.4% by weight, and a total THC content is at least 10.0% by weight, as measured by HPLC and calculated based on dry weight of the inflorescence.

In some embodiments, the cannabis plant, plant part, tissue or cell of the present invention may comprise a terpene oil content greater than 0.7% by weight wherein the terpene oil content is determined by the terpene profile as measured by HPLC, and calculated based on dry weight of the inflorescence. The terpene profile may comprise linalool, caryophyllene oxide, myrcene, beta-pinene, limonene, terpinolene, alpha-pinene, humulene, and caryolphyllene.

In some embodiments, the cannabis plant, plant part, tissue or cell of the present invention may comprise a terpene oil content greater than 2.1% by weight wherein the terpene oil content is determined by the terpene profile as measured by HPLC, and calculated based on dry weight of the inflorescence.

In other embodiments, the current application discloses a cannabis plant, plant part, tissue, or cell comprising: a myrcene relative content of less than 1.2% of the terpene profile, and a terpene oil content less than 2.1% by weight, wherein the terpene profile may comprise linalool, caryophyllene oxide, myrcene, beta-pinene, limonene, terpinolene, alpha-pinene, humulene, and caryolphyllene, and wherein the terpene oil content is determined by the additive content of the terpenes in the terpene profile, and wherein the terpene contents are measured by HPLC and calculated based on dry weight of the inflorescence.

In some embodiments, the cannabis plant, plant part, tissue or cell of the present invention may comprise a CBD content that is greater than 5% by weight as measured by HPLC and calculated based on dry weight of the inflorescence.

In some embodiments, the cannabis plant, plant part, tissue or cell of the present invention may comprise a THC content that is greater than 10% by weight as measured by HPLC and calculated based on dry weight of the inflorescence.

In other embodiments, the present invention teaches a cannabis plant, plant part, tissue, or cell comprising: a terpene oil content greater than 0.7% by weight, wherein the terpene profile may comprise linalool, caryophyllene oxide, myrcene, beta-pinene, limonene, terpinolene, alpha-pinene, humulene, and caryolphyllene, and wherein the terpene oil content is determined by the terpenes in the terpene profile, and wherein the cannabinoid and terpene contents are measured by HPLC and calculated based on dry weight of the inflorescence.

In some embodiments, the cannabis plant, plant part, tissue or cell of the present invention comprises a total cannabigerol (CBG) content that is greater than 0.05% as measured by HPLC and calculated based on dry weight of the inflorescence.

In some embodiments of the present invention, the cannabis plant comprises at least 0.02% CBC content by weight.

In some embodiments of the present invention, the first cannabis plant comprises a terpene oil content greater than 0.7% by weight wherein the terpene oil content is determined by the additive content of the terpenes in the terpene profile as measured by HPLC, and calculated based on dry weight of the inflorescence.

In some embodiments, the cannabis plant, plant part, tissue or cell of the present invention may have the following THC, CB and terpene profiles as measured by HPLC, and calculated based on dry weight of the inflorescence: 0.07% CBGA, <0.01% CBG, 0.06% CBG-TOTAL (CBGA* 0.878 +CBG), 13.24% Δ9-THC-A, 1.32% Δ9-THC, <0.01% Δ8-THC, <0.01% CBN, 12.93%, THC-TOTAL (Δ9-THC-A*0.877+Δ9THC), 7.59% CBDA, 0.23% CBD, 6.88% CBD-TOTAL, (CBDA*0.877+CBD), 0.04% CBC, with 1.59% ACTIVATED TOTAL and <0.01% Linalool, <0.01% Caryophyllene oxide, 0.67% Myrcene, <0.01% beta-Pinene, <0.01% Limonene, <0.01% Terpinolene, <0.01% alpha-Pinene, 0.62% Humulene, and 0.37% Caryophyllene with 1.66% TERPENE TOTAL. ACTIVATED TOTAL consists of Δ9-THC+Δ8-THC+CBN+CBD+CBG+CBC.

In some embodiments, the cannabis plant, plant part, tissue or cell of the present invention may have the following THC, CB and terpene profiles as measured by HPLC, and calculated based on dry weight of the inflorescence: 0.19% CBGA, <0.01% CBG, 0.17% CBG-TOTAL (CBGA*0.878+CBG), 10.50% Δ9-THC-A, 1.86% Δ9-THC, <0.01% Δ8-THC, <0.01% CBN, 11.07% THC-TOTAL (Δ9-THC-A*0.877+Δ9THC), 7.90% CBDA, 0.36% CBD, 7.28% CBD-TOTAL (CBDA*0.877+CBD), 0.05% CBC, with 2.27% ACTIVATED TOTAL and <0.01% Linalool, <0.01% Caryophyllene oxide, 0.78% Myrcene, <0.01% beta-Pinene, <0.01% Limonene, <0.01% Terpinolene, <0.01% alpha-Pinene, <0.01% Humulene, and <0.01% Caryophyllene, with 0.78% TERPENE TOTAL. ACTIVATED TOTAL consists of Δ9-THC+Δ8-THC+CBN+CBD+CBG+CBC.

In some embodiments, the cannabis plant, plant part, tissue or cell of the present invention may have the following THC, CB and terpene profiles as measured by HPLC, and calculated based on dry weight of the inflorescence: 0.11% CBGA, <0.01% CBG, 0.10% CBG-TOTAL (CBGA*0.878+CBG), 11.10% Δ9-THC-A, 0.70% Δ9-THC, <0.01% Δ8-THC, <0.01% CBN, 10.44% THC-TOTAL (Δ9-THC-A*0.877+Δ9THC), 5.96% CBDA, 0.25% CBD, 5.48% CBD-TOTAL (CBDA*0.877+CBD), 0.03% CBC, with 0.98% ACTIVATED TOTAL and <0.01% Linalool, <0.01% Caryophyllene oxide, 1.06% Myrcene, <0.01% beta-Pinene, <0.01% Limonene, <0.01% Terpinolene, 0.47% alpha-Pinene, 0.35% Humulene, and 0.15% Caryophyllene, with 2.03% TERPENE TOTAL. ACTIVATED TOTAL consists of Δ9-THC+Δ8-THC+CBN+CBD+CBG+CBC.

The invention claimed is:

1. A hybrid cannabis plant, or an asexual clone of said hybrid cannabis plant, or a plant part, tissue, or cell thereof, which produces an inflorescence, said inflorescence comprising of THC to CBD ratio of approximately 2:1.

2. A hybrid cannabis plant, or an asexual clone of said hybrid cannabis plant, or a plant part, tissue, or cell thereof, which produces an inflorescence, said inflorescence comprising a THC to CBD ratio of 1.9:1.

3. A hybrid cannabis plant, or an asexual clone of said hybrid cannabis plant, or a plant part, tissue, or cell thereof, which produces an inflorescence, said inflorescence comprising a THC to CBD ratio of 1.5:1.

4. A hybrid cannabis plant, or an asexual clone of said hybrid cannabis plant, or a plant part, tissue, or cell thereof, which produces an inflorescence, said inflorescence comprising a THC to CBD ratio of 2:1.

5. The hybrid cannabis plant, or an asexual clone of said hybrid cannabis plant, or a plant part, tissue, or cell thereof of claim 2, wherein the dominant terpene is myrcene.

6. The hybrid cannabis plant, or an asexual clone of said hybrid cannabis plant, or a plant part, tissue, or cell thereof of claim 2, wherein the dominant terpenes are myrcene, humulene, and caryophyllene.

7. The hybrid cannabis plant, or an asexual clone of said hybrid cannabis plant, or a plant part, tissue, or cell thereof of claim 2, wherein the dominant terpenes are myrcene, alpha-pinene, and cryophyllene.

8. The hybrid cannabis plant, or an asexual clone of said hybrid cannabis plant, or a plant part, tissue, or cell thereof of claim 3, wherein the dominant terpene is myrcene.

9. The hybrid cannabis plant, or an asexual clone of said hybrid cannabis plant, or a plant part, tissue, or cell thereof of claim 3, wherein CBD content is less than 3%.

10. The hybrid cannabis plant, or an asexual clone of said hybrid cannabis plant, or a plant part, tissue, or cell thereof of claim 2, wherein CBD content is less than 3%.

11. The hybrid cannabis plant, or an asexual clone of said hybrid cannabis plant, or a plant part, tissue, or cell thereof of claim 2, wherein THC content is less than 13%.

12. The hybrid cannabis plant, or an asexual clone of said hybrid cannabis plant, or a plant part, tissue, or cell thereof of claim 3, wherein THC content is less than 12%.
